(19) United States
(12) Patent Application Publication
(10) Pub. No.: US 2016/0093231 A1
(43) Pub. Date: Mar. 31, 2016

(54) APPARATUS AND METHOD FOR PROVIDING CONTENT FOR TRAINING NON-COGNITIVE ABILITY OF USER BY USING USER DEVICE

(71) Applicants: Joongang Edu, Seoul (KR); GRIT Research Institute, Seoul (KR)

(72) Inventors: Youngsan YOO, Seoul (KR); Joohan KIM, Seoul (KR)

(73) Assignees: GRIT RESEARCH INSTITUTE, Seoul (KR); JOONGANG EDU, Seoul (KR)

(21) Appl. No.: 14/557,837

(22) Filed: Dec. 2, 2014

(30) Foreign Application Priority Data
Sep. 30, 2014 (KR) 10-2014-0131497

(51) Int. Cl. G09B 19/00; A61B 5/117; A61B 5/0402; A61B 5/0476; G09B 5/02; A61B 5/0205

(52) U.S. Cl. CPC G09B 19/00; G09B 5/02; A61B 5/0205; A61B 5/0402; A61B 5/0476; A61B 5/117; A61B 5/02405

(57) ABSTRACT

Provided is a method of providing content for training a user's non-cognitive ability by using a user device, which includes acquiring user information of the user, performing user authentication based on the acquired user information, when the authentication succeeds, selecting one of a short-term training mode and a long-term training mode based on a user input and displaying at least one content according to the selected mode, and when the authentication fails, acquiring again updated user information. In this case, the at least one content is variably displayed according to the biometric information of the user.

### CROSS-REFERENCE TO RELATED APPLICATION

This application claims the benefit of Korean Patent Application No. 10-2014-0131497, filed on Sep. 30, 2014, in the Korean Intellectual Property Office, the entire disclosures of which are incorporated herein by reference for all purposes.

### BACKGROUND

1. Field of the Invention

One or more exemplary embodiments relate to an apparatus and method for providing content for training a user's non-cognitive ability.

2. Description of the Related Art

Personality tests have been considered as a kind of character test. In other words, personality tests are mainly intended to evaluate career consulting or aptitude, and factors handled by personality tests have no direct relation with user's personal achievement. In addition, no educational programs or solutions for enhancing personal achievement have been provided.

Recently, it has been found in positive psychology, positive organizational scholarship, human organization theory, and so on that a non-cognitive ability gives a direct and powerful influence to achievement. In particular, recent research results on the importance of GRIT have attracted various attentions. GRIT is the essence of the non-cognitive capacity, or the basis of motivation, tenacity, resilience, and relational competence. GRIT, the acronym for Growth Mindset, Resilience, Intrinsic Motivation, and Tenacity, is the source of “achievement competence,” or general human capacity. For students, the Grit Training System (GTS), will help with raising their grades and getting higher academic achievement. For adults, GTS will help with gaining higher task achievement, lowering stress levels, and strengthening mental muscles. GRIT, known to be “mental muscles,” can be developed and enhanced through regular and systematic trainings and “mental exercises.” GRIT test has a main objective of developing certain human personal traits to enhance their competency and achievement. GRIT test is intended to provide an objective understanding of individual characters and may measure an individual's non-cognitive ability in consideration of self-motivation, self-control, and interpersonal competency. GRIT test may further measure intrinsic motivation, autonomy, competency, growth mindset, efficiency, tenacity, passion for a goal, emotion control, positivity, empathic ability, relationship, loneliness, speech, and expressive power of a person to comprehensively evaluate and understand his/her character and then provide an educational program and solution according to a degree of development for each sub-characteristic. By using GRIT test, it is possible to understand the character objectively and comprehensively, find causes of various problems in daily life, and also find overall underachievement due to a character problem.

### SUMMARY

One or more exemplary embodiments include a method and apparatus for providing content for training a user's non-cognitive ability through a user device. In other words, it is possible to enhance the user's non-cognitive ability by determining a current degree of the user's non-cognitive ability and providing a variety of training content according to at least one of the determined level and type of the user's non-cognitive ability.

Additional aspects will be set forth in part in the description which follows and, in part, will be apparent from the description, or may be learned by practice of the presented exemplary embodiments.

According to one or more exemplary embodiments, a method of providing content for training a user's non-cognitive ability by using a user device includes acquiring user information of the user, performing user authentication based on the acquired user information, when the authentication succeeds, selecting any one of a short-term training mode and a long-term training mode based on a user input and displaying at least one piece of content according to the selected mode, and when the authentication fails, acquiring again updated user information, in which the at least one piece of content is variably displayed depending on the biometric information of the user.

The biometric information of the user may include at least one of a heart rate, a heart rate variability (HRV), a blood pressure, a respiration rate, an electrocardiogram (ECG), a galvanic skin response (GSR), and an electroencephalogram (EEG).

When the short-term training mode is selected, the displaying of the content may include displaying at least one image indicating at least one event that the user has a chance of encountering and displaying at least one piece of content mapped into an image selected from among the at least one displayed image according to an user input.

The method may further include acquiring the biometric information of the user while the content is displayed and analyzing the acquired biometric information, in which the at least one piece of content mapped into the selected image is intended to train the user's non-cognitive ability and variably provided depending on a result of the analysis of the biometric information.

When the long-term training mode is selected, the displaying of the content may include displaying at least one piece of test content for determining a level and type of the user's non-cognitive ability, determining the level and type of the user's non-cognitive ability based on a user input with respect to the test content, setting a training condition according to the determined level and type, and displaying the at least one piece of content for training the user's non-cognitive ability according to the set training condition, in which the training condition includes at least one of a kind of the displayed content, a display time, and a display scheme.

The method may further include acquiring the biometric information of the user while the content is displayed, analyzing the acquired biometric information, and resetting the training condition according to a result of the analysis of the biometric information, in which the at least one piece of content for training the user's non-cognitive ability is displayed based on the reset training condition.

According to one or more exemplary embodiments, an apparatus for providing content for training a non-cognitive ability of a user by using a user device includes an external-input reception unit configured to acquire user information of the user and receive an input applied by the user, an authentication unit configured to perform user authentication based on the acquired user information, a mode selection unit configured to select any one of a short-term training mode and a long-term training mode based on the user input, and a display unit configured to display at least one piece of content according to the selected mode, in which updated user information is acquired again through the external-input reception unit when the authentication fails, and the at least one of the content is variably displayed depending on the biometric information of the user.

When the short-term training mode is selected, the display unit may display at least one image indicating at least one event that the user has a chance of encountering and display at least one piece of content mapped into an image selected from among the at least one displayed image according to an user input received through the external-input reception unit.

The apparatus may further include a biometric-information acquisition unit configured to acquire the biometric information of the user while the content is displayed and an analysis unit configured to analyze the acquired biometric information, in which the at least one piece of content mapped into the selected image is intended to train the user's non-cognitive ability and variably provided depending on a result of the analysis of the biometric information.

The apparatus may further include a determination unit configured to determine a level and type of the user's non-cognitive ability and a training condition setting unit configured to set a training condition according to the determined level and type, in which, when the long-term training mode is selected, the display unit displays at least one piece of test content for determining the level and type of the user's non-cognitive ability, the determination unit determines the level and type of the user's non-cognitive ability based on a user input with respect to the test content received through the external-input reception unit, the training condition setting unit sets the training condition according to the determined level and type, the display unit displays at least one piece of content for training the user's non-cognitive ability according to the set training condition, and the training condition includes at least one of a kind of the displayed content, a display time, and a display scheme.

The apparatus may further include a biometric-information acquisition unit configured to acquire biometric information of the user while the content is displayed and an analysis unit configured to analyze the acquired biometric information, in which the training condition is reset by the training condition setting unit according to a result of the analysis of the biometric information and at least one piece of content for training the user's non-cognitive ability is displayed through the display unit on the basis of the reset training condition.

According to one or more exemplary embodiments, a computer-readable recording medium reads a computer program for executing the method.

### BRIEF DESCRIPTION OF THE DRAWINGS

These and/or other aspects will become apparent and more readily appreciated from the following description of the exemplary embodiments, taken in conjunction with the accompanying drawings in which:

FIG. 1 is a schematic view illustrating a process of providing content for training a user's non-cognitive ability according to an embodiment of the present invention;

FIG. 2 is a flowchart showing a method of providing content for training a user's non-cognitive ability according to an embodiment of the present invention;

FIG. 3 shows a method of displaying content when a short-term training mode is selected according to an embodiment of the present invention;

FIG. 4 is a flowchart showing a method further including acquiring and analyzing biometric information according to an embodiment of the present invention;

FIG. 5A shows a method of displaying content when a long-term training mode is selected according to an embodiment of the present invention;

FIGS. 5B to 5C show examples of the test content that may be provided to a user according to an embodiment of the present invention;

FIGS. 6A to 6F show examples of displaying information on a level and type of the user's non-cognitive ability according to an embodiment of the present invention;

FIG. 7 is a flowchart showing a method further including acquiring and analyzing biometric information according to another embodiment of the present invention; and

FIG. 8 is a block diagram showing an apparatus for providing content for training a user's non-cognitive ability according to an embodiment of the present invention.

### DETAILED DESCRIPTION

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. Accordingly, the exemplary embodiments are merely described below, by referring to the figures, to explain aspects of the present description. Expressions such as “at least one of,” when preceding a list of elements, modify the entire list of elements and do not modify the individual elements of the list.

Terms used herein will be briefly described, and the present invention will be described in detail.

Terms used in the present invention have been selected as general terms which are widely used at present, in consideration of the functions of the present invention, but may be altered according to the intent of an operator of ordinary skill in the art, conventional practice, or introduction of new technology. Also, if there is a term which is arbitrarily selected by the applicant in a specific case, in which case a meaning of the term will be described in detail in a corresponding description portion of the present invention. Therefore, the terms should be defined on the basis of the entire contents of this specification instead of a simple name of each of the terms.

In this disclosure below, when it is described that one comprises (or includes or In this disclosure below, when it is described that one comprises (or includes or has) some elements, it should be understood that it may comprise (or include or has) only those elements, or it may comprise (or include or have) other elements as well as those elements if there is no specific limitation. Moreover, each of terms such as “unit” and “module” described in specification denotes an element for performing at least one function or operation, and may be implemented in hardware, software or the combination of hardware and software. In addition, each of terms such as “present” and “provide” described in association with the description of the present invention denotes a method for expressing data that may be delivered from the apparatus to the user, for example, display of an image, output of a voice, and vibration during a certain time. The present invention is not limited thereto.

Hereinafter, embodiments of the present invention will be described in detail to be easily embodied by those of ordinary skill in the art with reference to the accompanying drawings. The present invention may, however, be embodied in many different forms and should not be construed as being limited to the embodiments set forth herein. In the accompanying drawings, a portion irrelevant to a description of the present invention will be omitted for clarity. Moreover, like reference numerals refer to like elements throughout.

Human abilities may include cognitive elements and non-cognitive elements.

Non-cognitive ability may include self-control which is an ability to make an effort for himself/herself, self-motivation which is an ability to make a motivation for themselves, and interpersonal competency which is an ability to have and keep good relations with other persons.

The non-cognitive ability is mental strength that enables tenacity, passion, and sociability and may be a driving force for achievement. Many studies have revealed that the non-cognitive ability can be enhanced by repetitive mental training, like a muscle of a body. In particular, core training for enhancing self-control and self-motivation is positive sentiment inducing training.

In this specification, GRIT may be a concept that is used to collectively refer to non-cognitive abilities such as growth mindset, resilience, intrinsic motivation, and tenacity. Self-motivation may include growth mindset and intrinsic motivation, and self-control may include resilience and tenacity. To enhance the grit, habituation is needed such that a brain may more positively respond to the same stimulation (for example, situation). In other words, more powerful grit may be developed through repetitive training for inducing positive feeling. For example, there are research results showing that the positive-feeling training may deactivate amygdala and activate prefrontal lobe For example, there are many research results showing that the activation of amygdala, which is a source that induces negative feelings, such as uneasiness, fear, and anger, is reduced and a specific region (for example, ventrolateral prefrontal cortex (VLPFC)) of prefrontal lobe, which is a source for self-control and rational decision, is activated when a meditation training or a training of naming own emotions are performed.

Several methods are known as a method for enhancing the grit. For example, the method may refer to the positive-feeling training. In order to show the grit, which is mental strength, people should be healthy and competent. The repetitive training for inducing positive feelings makes people more healthy and competent. In addition, the training for inducing positive feelings may strengthen immunity and reduce stress to prevent cardiovascular diseases.

Examples of the training for inducing positive feelings may include a gratitude meditation, a love-kindness meditation, thinking, speaking, and writing of own strengths and merits, a good and kind conduct, writing of a gratitude diary or note, confessing respect, savoring, growth mindset training, and freedom from other people's eyes.

According to an embodiment of the present invention, it is possible to strengthen or maintain the non-cognitive ability by providing the user with a variety of content for the above-described positive-feeling training such that the user may use the content freely.

Hereinafter, embodiments of the present invention will be described in detail with reference to the accompanying drawings.

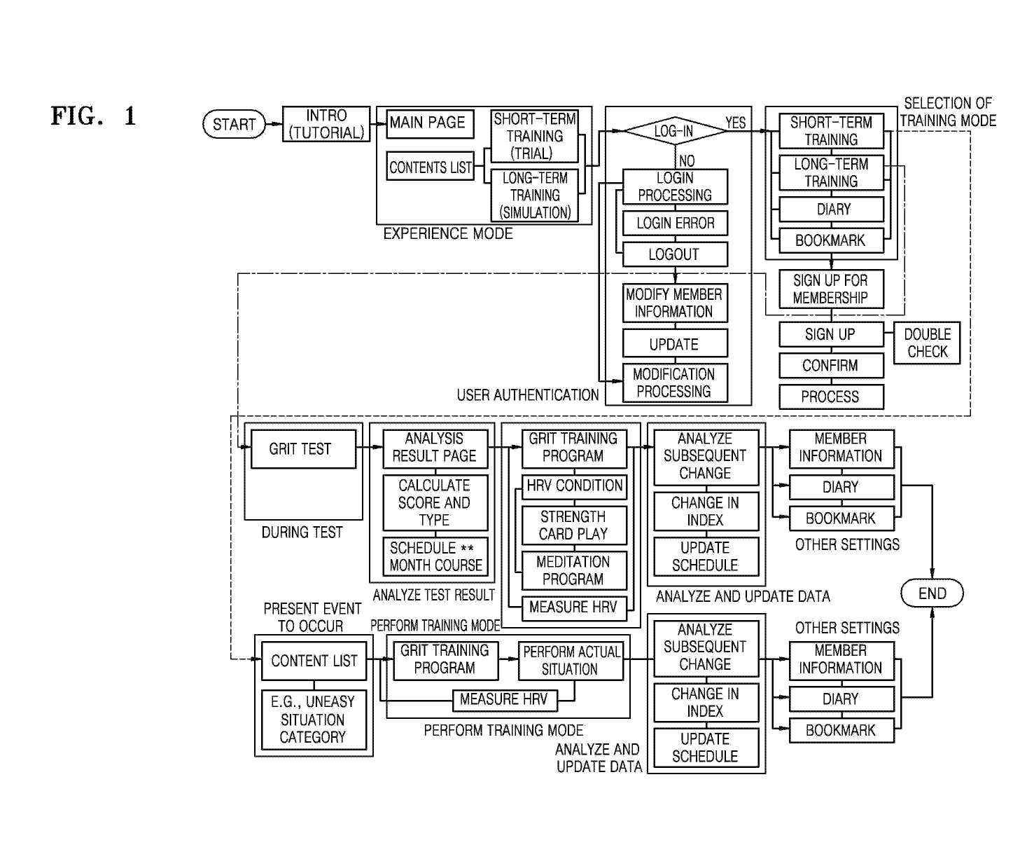

FIG. 1 is a schematic view illustrating a process of providing content for training a user's non-cognitive ability according to an embodiment of the present invention.

The process of providing content for training a non-cognitive ability according to an embodiment of the present invention may include an introduction stage for providing a description about the non-cognitive ability training, a description about a procedure of the training, and the like. In addition, the process may include an experience mode stage to enable an experience of the training before user authentication. The experience mode stage may include a short-term training experience, a long-term training simulation, and the like. Everyone may receive content for non-cognitive training on a trial basis through the experience mode, thus increasing a user's convenience in using a service.

A user authentication stage for the non-cognitive training may be required according to an embodiment of the present invention. Information on a training history and mental and physical conditions of a user may be encrypted and managed through the user authentication. The user authentication may be performed by determining whether identification information of a user, for example, an ID and a password, that is previously established in a storage unit (not shown) is consistent with user information received through a user input unit. The user information may include a name, a birth date, a gender, an affiliation, and login information (for example, identification information). When the user authentication fails, the user information is received again from the user and the authentication stage may be performed again. As described above, through the user authentication, the user may selectively read records stored with respect to his/her training history and mental and physical conditions. In addition, the user may modify his/her user information, and the modified user information may be stored by updating data in the storage unit (not shown).

The user may select and use any one of the short-term training mode and the long-term training mode according to an embodiment of the present invention. In other words, a certain menu for selecting the training mode may be provided to the user according to an embodiment of the present invention. The menu may be in the form of a list including a functional icon. For example, the user may select the short-term training mode by pressing an icon indicating the short-term training mode on a display unit. In addition, the menu may be output through a sound output unit (not shown) in the form of a voice and the like.

When the short-term training mode is selected, an image for indicating at least one event that the user has a chance of encountering may be displayed as an embodiment of the present invention. The at least one event may include various uneasy situations, for example, an uneasy feeling before taking an examination, making a presentation, giving birth, and attending a conference. In other words, in order for the user to reduce the uneasy feeling that may be experienced when the event occurs, the non-cognitive ability training may be needed according to an embodiment of the present invention.

As an embodiment of the present invention, according to an input for the user to select an image (for example, a test application image) from among displayed images (for example, a test application image, a presentation image, a birth image, and the like), at least one piece of content that is mapped into an image (for example, a test application image) may be displayed. The at least one piece of content may include a positive-meaning word (for example, being good at concentrating or solving problems, etc.) a concentration-enhancing sound, and the like.

Biometric information of the user may be acquired according to an embodiment of the present invention. The biometric information of the user are measured and acquired during all day (24 hours) or during a certain period of time (for example, during the training). The biometric information of the user may include at least one of a heart rate, a heart rate variability (HRV), a blood pressure, a respiration rate, an electrocardiogram (ECG), a galvanic skin response (GSR), and an electroencephalogram (EEG). The biometric information may be included in an apparatus according to an embodiment of the present invention or in a device that is separately located outside the apparatus and configured to perform wired or wireless communication with the apparatus.

The biometric information of the user may be acquired while the content is displayed according to an embodiment of the present invention. The acquired biometric information may be analyzed based on various factors. For example, the various factors may include an index such as a root mean square of successive difference (RMSSD) and a standard deviation of N-N interval (SDNN) that are acquired through the analysis of the heart rate variability (HRV).

At least one piece of content that is mapped into the selected image according to an embodiment of the present invention is intended to train a user's non-cognitive ability, and may be provided variably depending on a result of the analysis of the biometric information. For example, a frequency of a positive-meaning word, a concentration-enhancing sound, or the like may increase or decrease according to the heart rate variability (HRV) of the user.

In addition, as described above, the biometric information may be measured and acquired continuously during daily life of the user after the completion of the training, as well as during the training.

According to an embodiment of the present invention, data indicating mental and physical conditions of the user may be acquired using a result of training the user's non-cognitive ability and the acquired biometric information. In other words, during and after the training, it may be determined which kind or type of training is subsequently performed by the user through a comprehensive analysis of the non-cognitive ability training result for the user and the biometric information of the user. A training schedule of the user may be updated.

In addition, the training result for the user may be stored in the storage unit (not shown) in combination with user information.

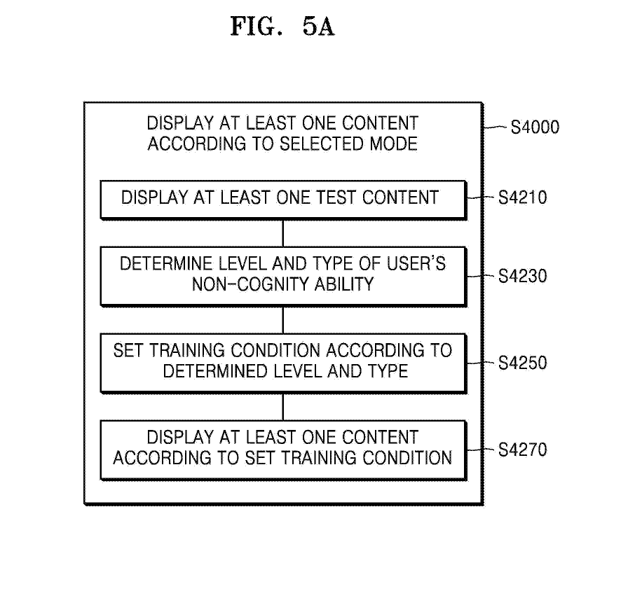

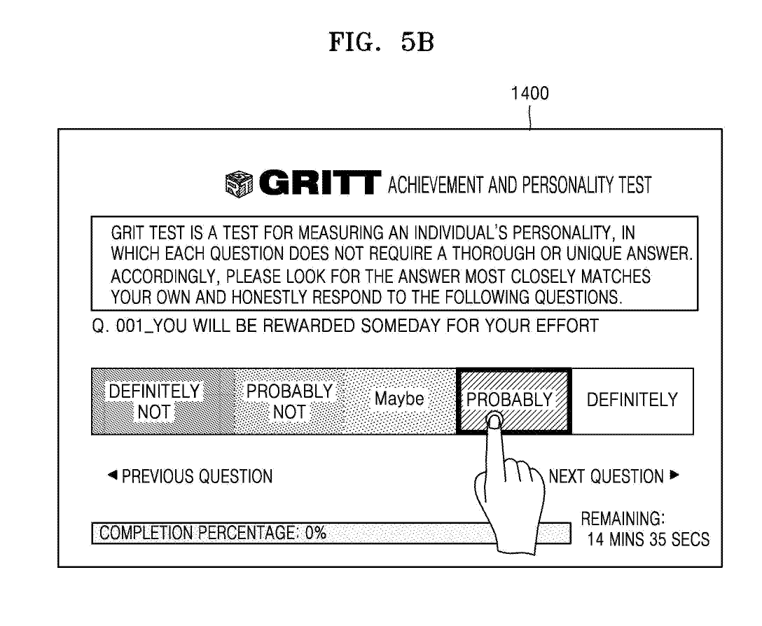

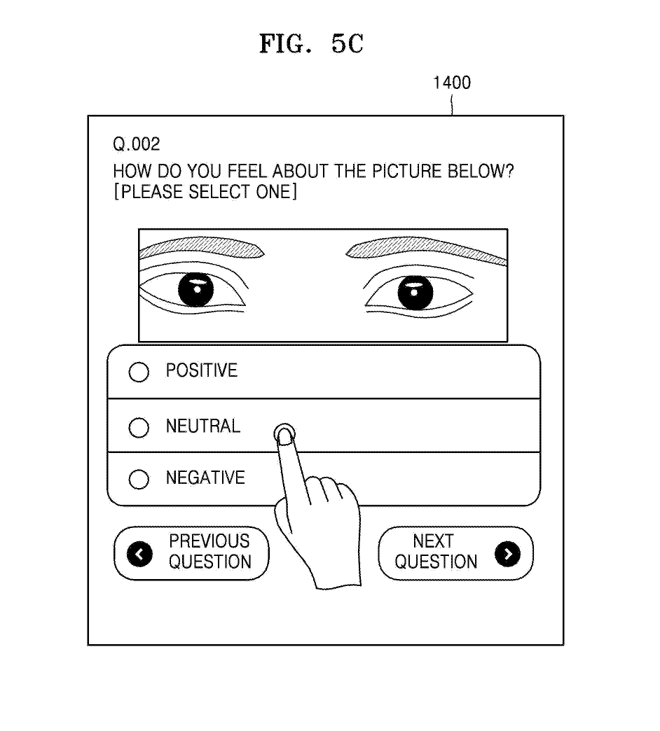

Furthermore, when the long-term training mode is selected, at least one piece of test content used to determine the level and type of the user's non-cognitive ability may be displayed. The provision of the test content may be referred to as a grit test. The grit test may be performed by presenting questions on a basis of various factors. The test content may be provided through, but not limited to, a display unit 1400 as shown in FIGS. 5B to 5C. For example, the grit test may be performed in a question and answer form of providing at least one sentence, image, and sound to a user as a test question and receiving a response to the provided at least one question from the user.

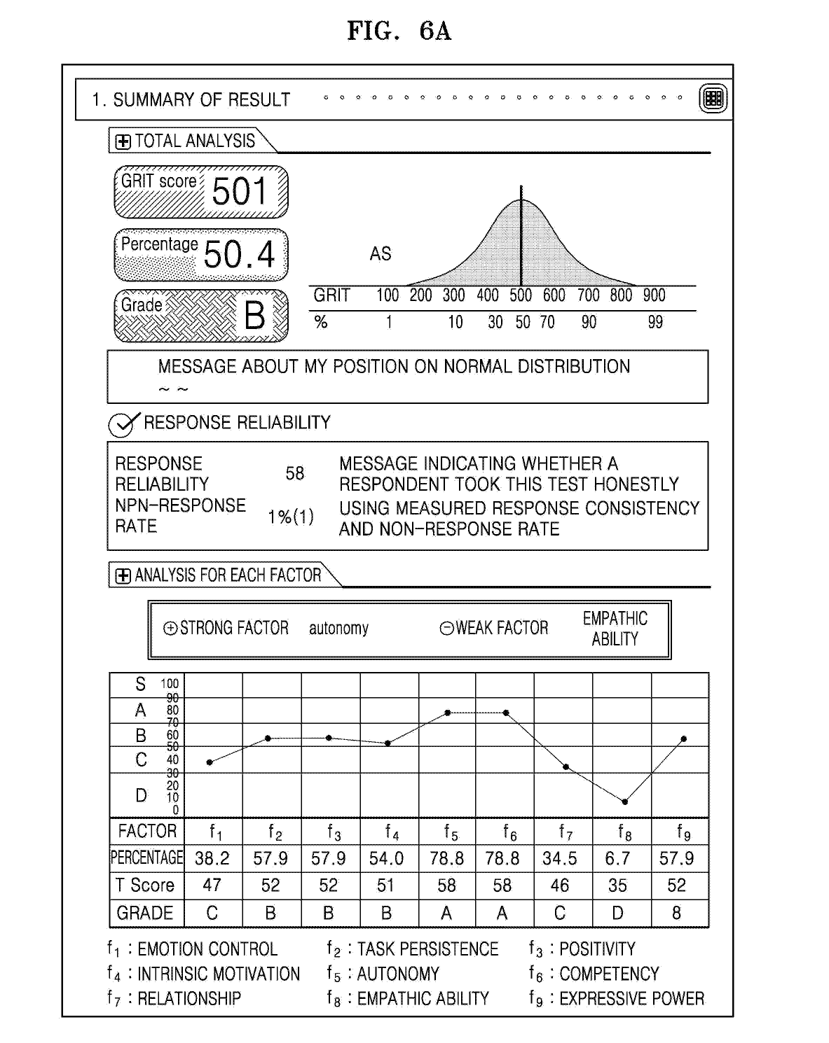

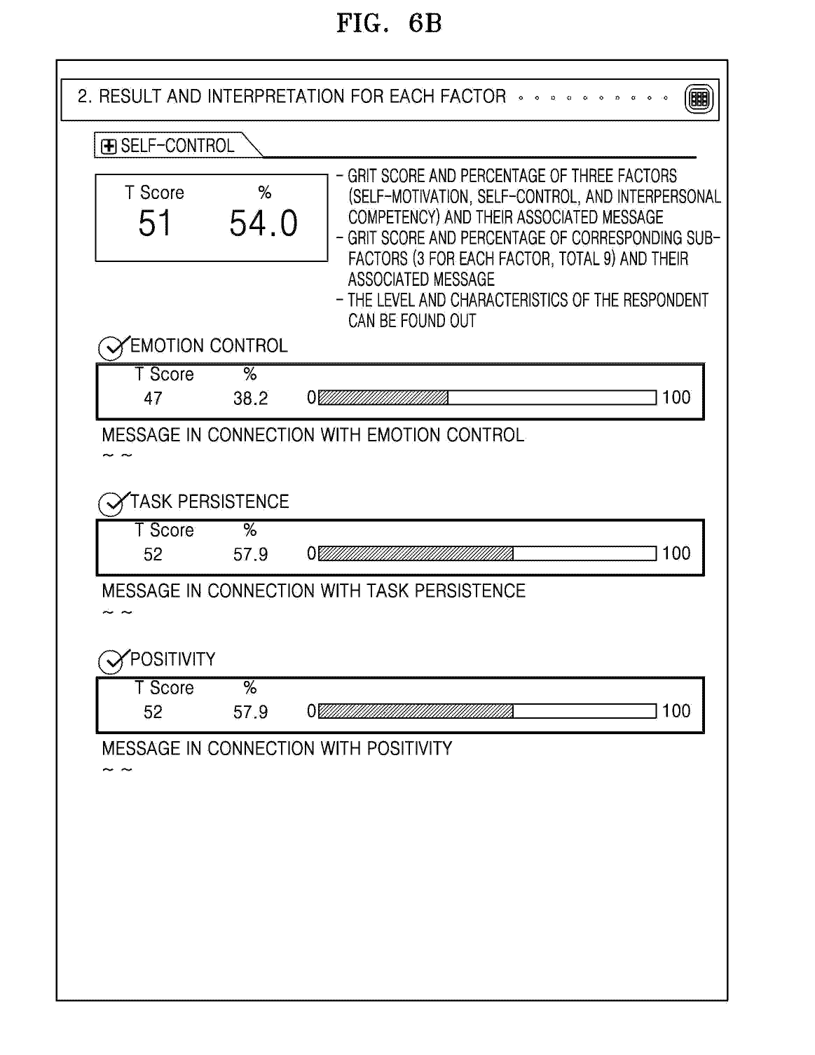

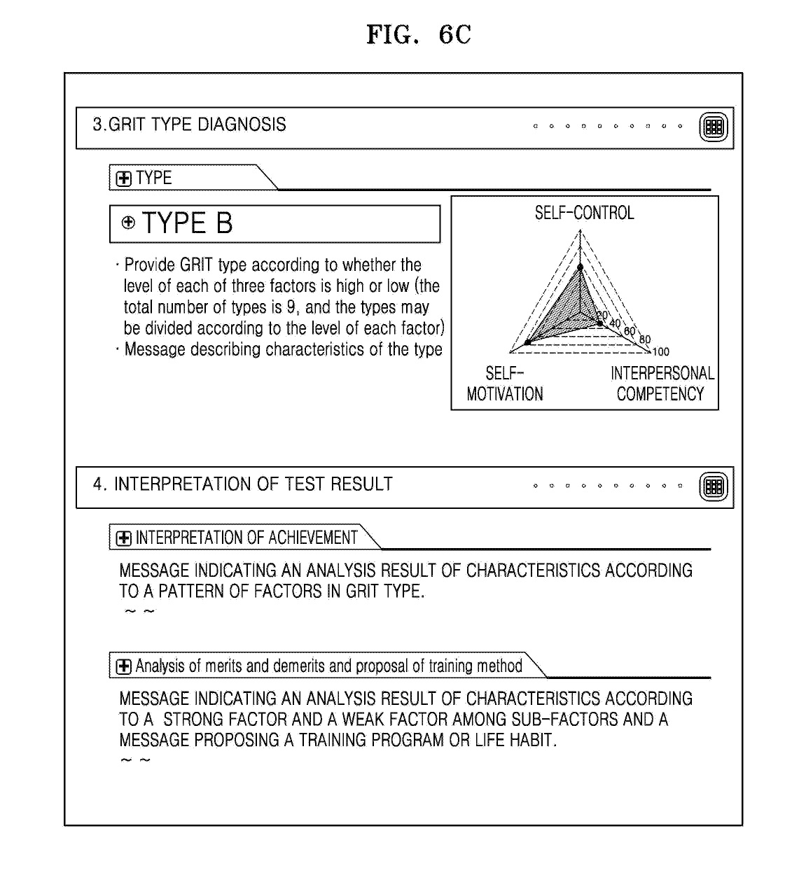

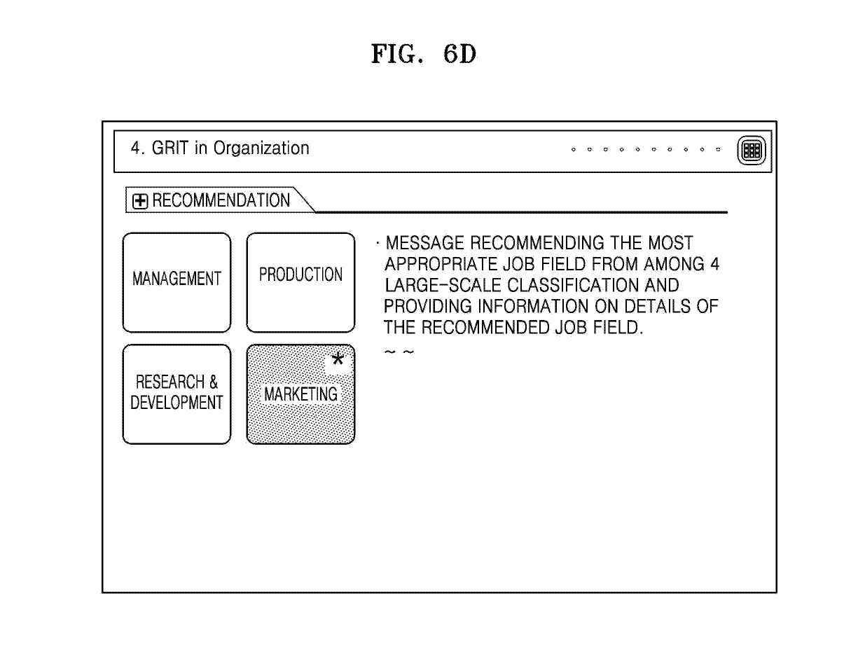

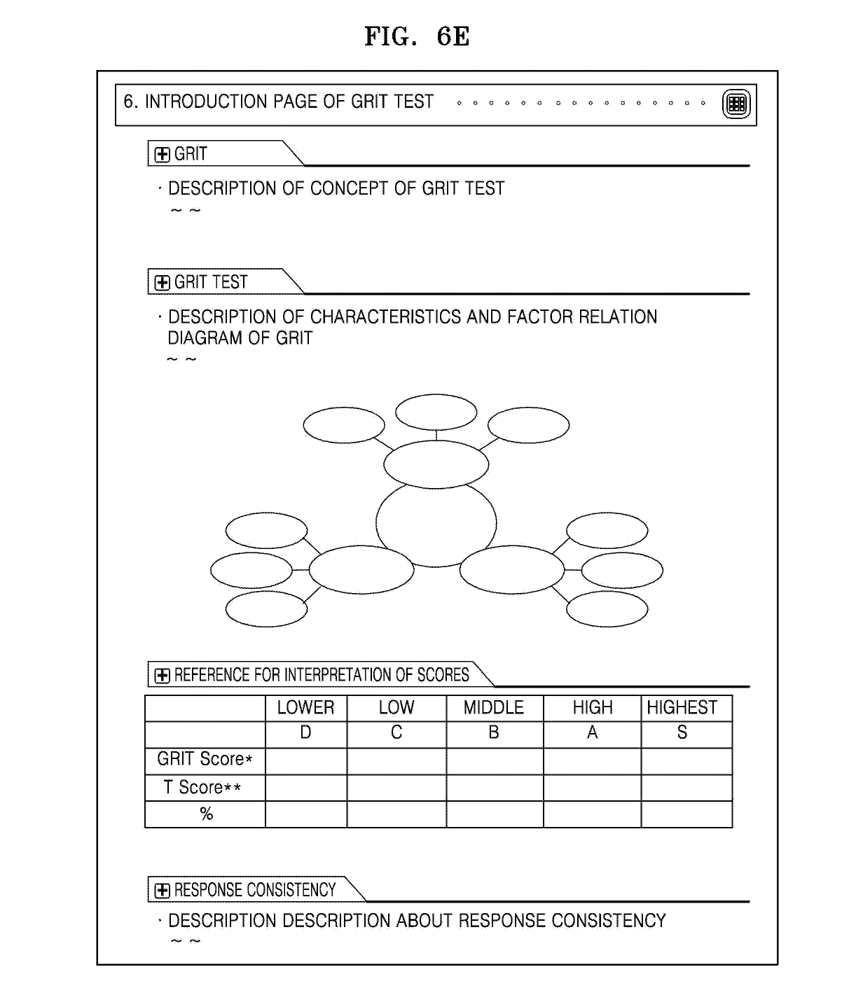

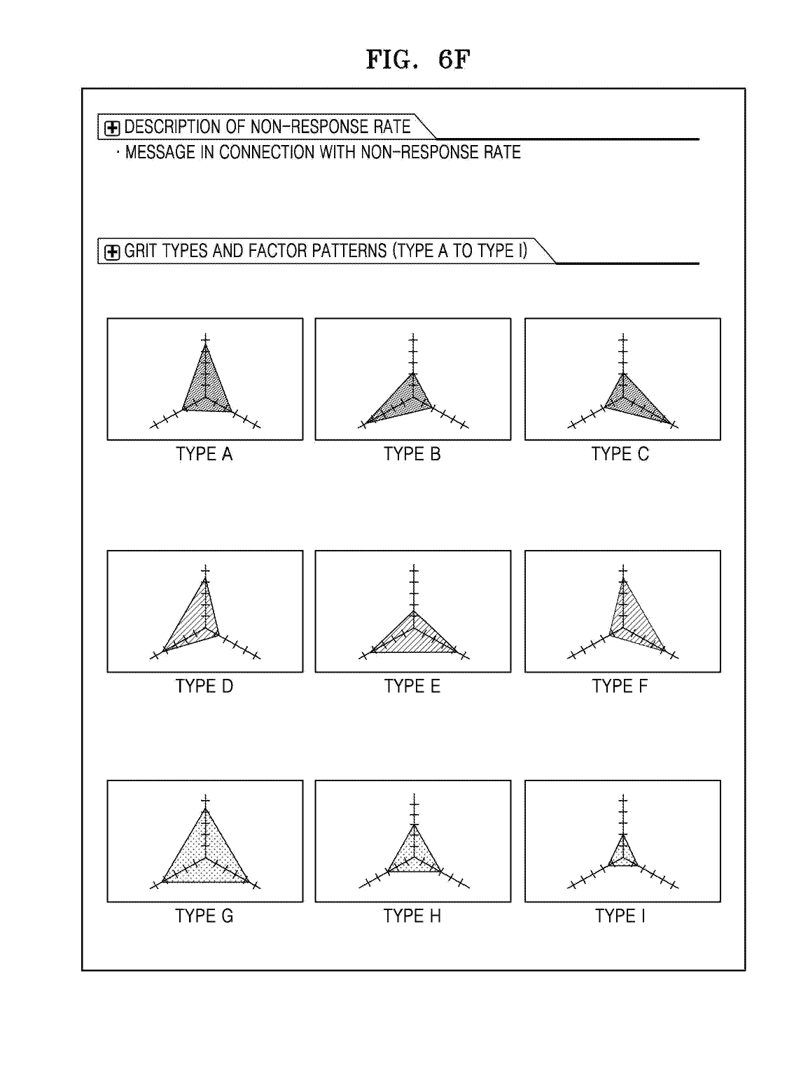

According to an embodiment of the present invention, the level and type of the user's non-cognitive ability may be determined on the basis of a user input with respect to the test content. For example, the non-cognitive ability result for the user may be represented as shown in FIG. 6A to 6F.

A training condition may be set according to the determined level and type. The training condition may include at least one of a training period, a kind of the displayed content, a display time, and a display scheme. The training condition may be referred to as a training schedule or a schedule.

In addition, at least one piece of content for training a user's non-cognitive ability may be displayed according to the set training condition. As described above, the content may include an audio or video, such as a positive-meaning word and a concentration enhancing sound. For example, positive-meaning words may be presented to a user as a strength card play, or at least one of a sound and an image for relaxing a body and refreshing a mind may be presented to a user as a meditation program.

In addition, as described above, the biometric information may be measured and acquired continuously during daily life of the user after completion of the training, as well as during the training, data indicating mental and physical conditions of the user may be acquired using the non-cognitive ability training result for the user and the acquired biometric information of the user, and a training schedule of the user may be updated.

The above-described menu may include an icon for a diary function, an icon for a bookmark function, and the like in addition to a functional icon for selecting a training mode. With the diary function, the user may check at once information for use, such as a daily training score result, a daily change in index, a position in a candidate group, a training program schedule, and a training program completion rate. In addition, with the bookmark function, the user may previously register a training program or menu that is frequently used by the user, thus easily approaching the menu according to selection of the user.

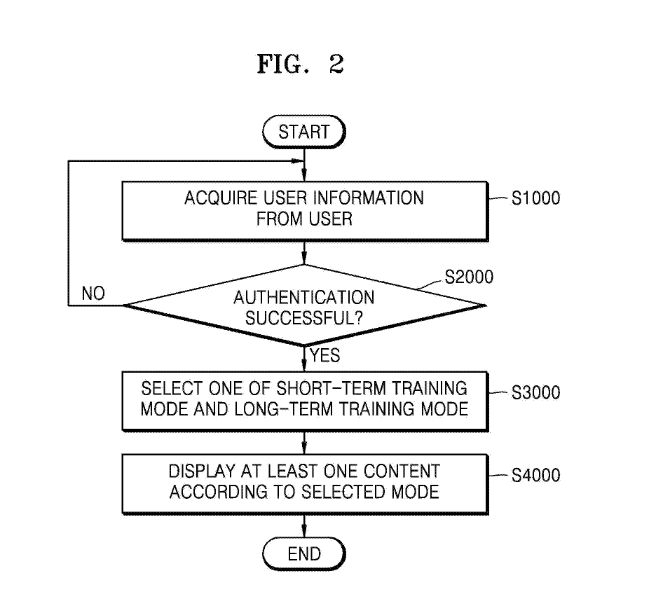

FIG. 2 is a flowchart showing a method of providing content for training a user's non-cognitive ability according to an embodiment of the present invention.

A method of providing content for training a user's non-cognitive ability by using a user device according to an embodiment of the present invention may include acquiring user information of a user (S1000), performing user authentication based on the acquired user information (S2000), selecting any one of a short-term training mode and a long-term training mode based on a user input when the authentication succeeds (S3000), and displaying at least one piece of content according to the selected mode (S4000). When the authentication fails, updated user information may be acquired again. Biometric information of the user may include at least one of a heart rate, a heart rate variability (HRV), a blood pressure, and a respiration rate.

The at least one piece of content may be displayed variably depending on the biometric information of the user. For example, a frequency of a positive-meaning word, a concentration-enhancing sound, or the like may increase or decrease according to the heart rate variability (HRV) of the user.

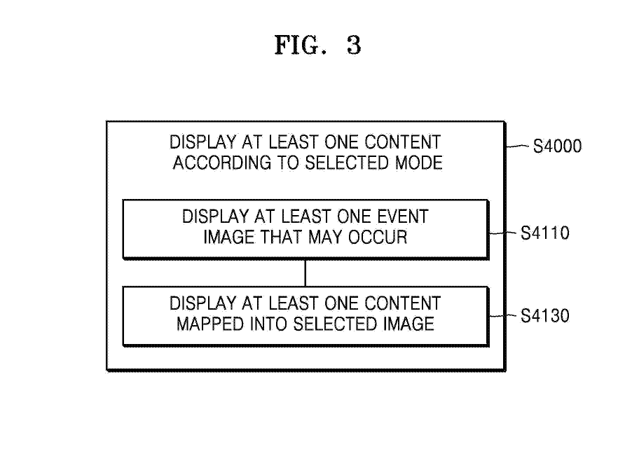

FIG. 3 shows a method of displaying content when a short-term training mode is selected according to an embodiment of the present invention.

When the short-term training mode is selected, the displaying of the content (S4000) may include displaying an image indicating at least one event that the user has a chance of encountering (S4110) and displaying at least one piece of content mapped into an image selected from among the at least one displayed image according to an user input (S4130).

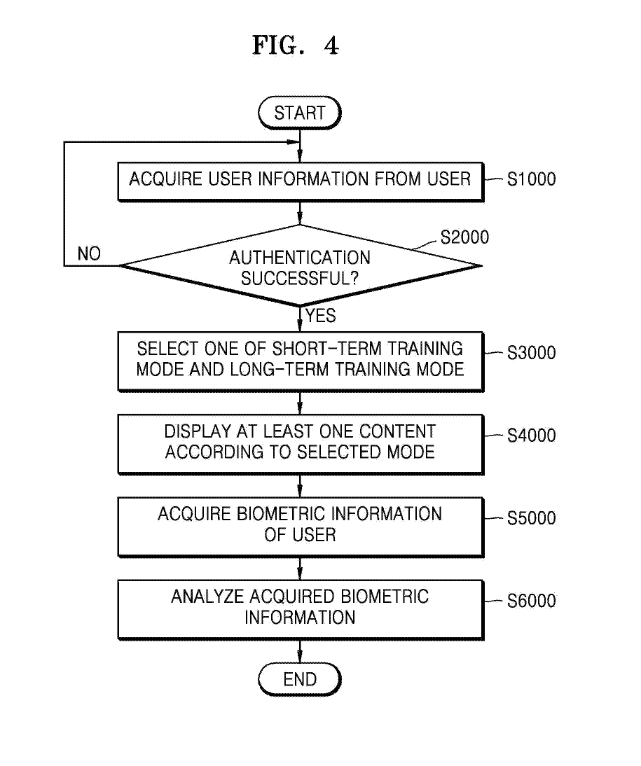

FIG. 4 is a flowchart showing a method further including acquiring and analyzing biometric information according to an embodiment of the present invention.

The method according to an embodiment of the present invention may further include acquiring biometric information of the user while the content is displayed (S5000) and analyzing the acquired biometric information (S6000). At least one piece of content that is mapped into the selected image is intended to train a user's non-cognitive ability, and may be provided variably depending on a result of the analysis of the biometric information.

FIG. 5A shows a method of displaying content when a long-term training mode is selected according to an embodiment of the present invention.

When the long-term training mode is selected according to an embodiment of the present invention, the displaying of the content (S4000) may include displaying at least one piece of test content for determining a level and type of the user's non-cognitive ability (S4210), determining the level and type of the user's non-cognitive ability based on a user input with respect to the test content (S4230), setting a training condition according to the determined level and type (S4250), and displaying the at least one piece of content for training the user's non-cognitive ability according to the set training condition (S4270). The training condition may include at least one of a training period, a kind of the displayed content, a display time, and a display scheme.

FIGS. 5B to 5C show examples of the test content that may be provided to a user according to an embodiment of the present invention.

At least one of a level and type of the user's non-cognitive ability may be determined the provision of the test content, which is referred to as a grit test. As described above, the grit test may be conducted by presenting questions on a basis of various factors through the display unit 1400 and the like. In other words, at least one piece of content for the grit test may be provided, as shown in FIGS. 5B to 5C. The present invention is not limited thereto. For example, the content providing method according to an embodiment of the present invention may determine the level and type of the user's non-cognitive ability by providing at least one sentence, image, sound, and the like to a user as at least one test question through the grit test, receiving a user response to the provided at least one test question, and analyzing characteristics of the received user response.

FIGS. 6A to 6F show examples of displaying information on the level and type of the user's non-cognitive ability determined according to an embodiment of the present invention.

The level and type of the user's non-cognitive ability may be determined according to an external input applied by the user in response to the test content. For example, the user may respond to the test content, such as questions on a basis of various factors. The response result may be analyzed and represented as the level and type of the user's non-cognitive ability. The factors may include, for example, emotion control, task persistence, positivity, intrinsic motivation, autonomy, competency, relationship, empathic ability, and expressive power. Questions for checking the factors may be previously set and stored in the storage unit (not shown).

As shown in FIG. 6A, GRIT score, a percentage, a grade based on a percentage, and the like may be comprehensively presented to a user. In addition, an analysis for each factor may be represented as a score, a percentage, a grade, and the like.

As shown in FIG. 6B, a result of the factor-based analysis may be presented in detail. Moreover, as shown in FIG. 6C, a type of the user's non-cognitive ability may be determined and presented. The type of the user's non-cognitive ability may be classified, for example, as shown in FIG. 6F.

In addition, a comprehensive analysis of the test result may be presented, as shown in FIG. 6C, and a job field suitable for a user may be recommended, as described in FIG. 6D.

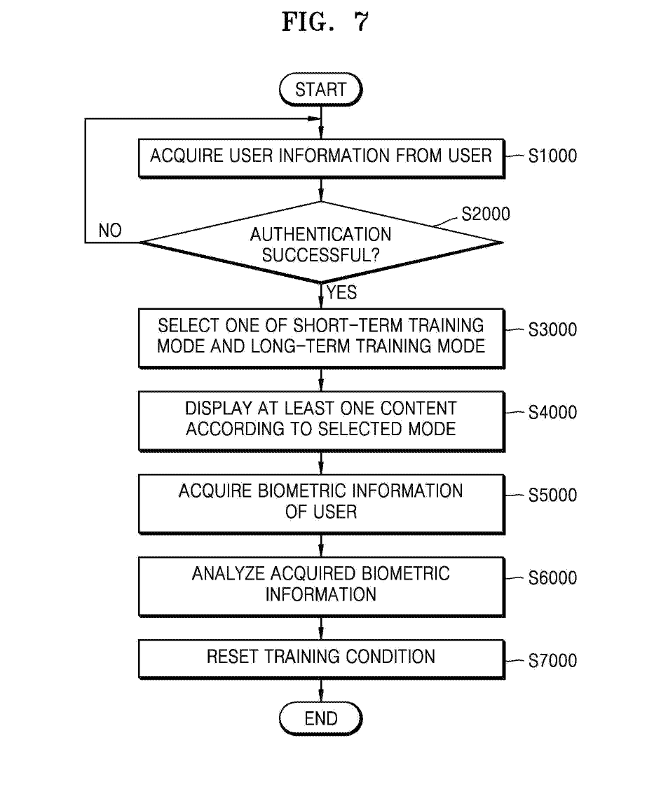

FIG. 7 is a flowchart showing a method further including acquiring and analyzing biometric information according to another embodiment of the present invention.

The method according to an embodiment of the present invention may further include acquiring biometric information of the user while the content is displayed (S5000), analyzing the acquired biometric information (S6000), and resetting a training condition according to a result of the analysis of the biometric information (S7000). At least one piece of content for training a user's non-cognitive ability may be displayed based on the reset training condition. In other words, the at least one piece of content may be displayed according to an updated training schedule.

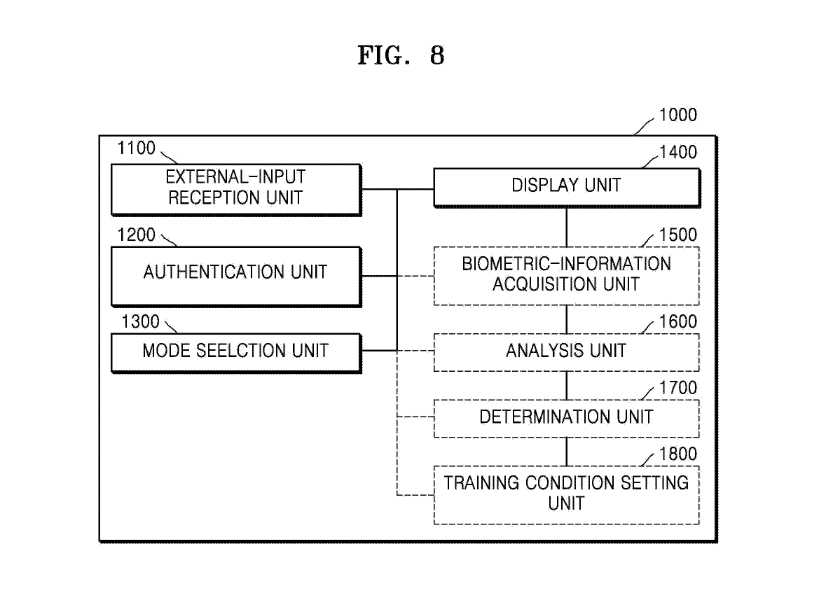

FIG. 8 is a block diagram showing an apparatus for providing content for training a user's non-cognitive ability according to an embodiment of the present invention.

An apparatus 1000 for providing content for training a user's non-cognitive ability by using a user device according to an embodiment of the present invention may include an external-input reception unit 1100 configured to acquire user information of the user and receive an input applied by the user, an authentication unit 1200 configured to perform user authentication based on the acquired user information, a mode selection unit 1300 configured to select any one of a short-term training mode and a long-term training mode based on the user input, and a display unit 1400 configured to display at least one piece of content according to the selected mode. The external-input reception unit 1100 according to an embodiment of the present invention may include a device such as a touch panel, a mouse, a keyboard, a microphone, and the like.

The apparatus according to an embodiment of the present invention acquires updated user information through the external-input reception unit 1100 when the authentication fails. The at least one piece of content may be displayed variably depending on the biometric information of the user. For example, the apparatus 1000 according to an embodiment of the present invention may display a message for requesting the user to input user information again through the display unit 1400 when the authentication fails.

The biometric information of the user may include at least one of a heart rate, a heart rate variability (HRV), a blood pressure, and a respiration rate.

When the short-term training mode is selected, the display unit 1400 may display an image indicating at least one event that the user has a chance of encountering and display at least one piece of content mapped into an image selected from among the at least one displayed image according to an user input received through the external-input reception unit 1100.

The apparatus 1000 according to an embodiment of the present invention may further include a biometric-information acquisition unit 1500 configured to acquire the biometric information of the user while the content is displayed and an analysis unit 1600 configured to analyze the acquired biometric information. At least one piece of content that is mapped into the selected image is intended to train a user's non-cognitive ability, and may be provided variably depending on a result of the analysis of the biometric information. The biometric-information acquisition unit 1500 may be a device including all kinds of sensors for measuring the biometric information such as a heart rate. For example, the biometric-information acquisition unit 1500 may include a wearable device such as a smart watch.

Furthermore, the apparatus according to an embodiment of the present invention may further include a determination unit 1700 configured to determine a level and type of the user's non-cognitive ability and a training condition setting unit 1800 configured to set a training condition according to the determined level and type. When the long-term training mode is selected, the display unit 1400 displays at least one test content for determining the level and type of the user's non-cognitive ability, the determination unit 1700 determines the level and type of the user's non-cognitive ability based on a user input with respect to the test content received through the external-input reception unit 1100, and the display unit 1800 sets a training condition according to the determined level and type and displays the at least one piece of content for training the user's non-cognitive ability according to the set training condition through the display unit 1400. The training condition may include at least one of a training period, a kind of the displayed content, a display time, and a display scheme.

The apparatus 1000 according to an embodiment of the present invention may further include a biometric-information acquisition unit 1500 configured to acquire biometric information of the user while the content is displayed and an analysis unit 1600 configured to analyze the acquired biometric information, in which the training condition is reset by the training condition setting unit 1800 according to a result of the analysis of the biometric information, and at least one piece of content for training the user's non-cognitive ability may be displayed through the display unit 1400 on the basis of the reset training condition.

The above-described method may be applied to the apparatus according to an embodiment of the present invention. Accordingly, repetitive description on the above-described method will be omitted.

As described above, according to the one or more of the above exemplary embodiments, a method and apparatus for providing content for training a user's non-cognitive ability through a user device are provided. In other words, it is possible to enhance the user's non-cognitive ability by determining a current degree of the user's non-cognitive ability and providing a variety of training content according to the determined level and type of the user's non-cognitive ability.

Furthermore, it is also possible to stabilize mental and physical conditions of the user and strengthen the non-cognitive ability of the user by providing at least one piece of content that is beneficial to the user in various user situations, for example, an uneasy situation such as before a test and before childbirth.

It should be understood that the exemplary embodiments described therein should be considered in a descriptive sense only and not for purposes of limitation. Descriptions of features or aspects within each exemplary embodiment should typically be considered as available for other similar features or aspects in other exemplary embodiments.

While one or more exemplary embodiments have been described with reference to the figures, it will be understood by those of ordinary skill in the art that various changes in form and details may be made therein without departing from the spirit and scope as defined by the following claims.

What is claimed is:

1. An apparatus for providing content for training a non-cognitive ability of a user by using a user device, the apparatus comprising:
an external-input reception unit configured to acquire user information of the user and receive an input applied by the user;
an authentication unit configured to perform user authentication based on the acquired user information;
a mode selection unit configured to select one of a short-term training mode and a long-term training mode based on the user input; and
a display unit configured to display at least one content according to the selected mode,
wherein updated user information is acquired again through the external-input reception unit when the authentication fails, and the at least one content is variably displayed according to the biometric information of the user.

2. The apparatus of claim 1, wherein the biometric information of the user comprises at least one of a heart rate, a heart rate variability (HRV), a blood pressure, a respiration rate, an electrocardiogram (ECG), a galvanic skin response (GSR), and an electroencephalogram (EEG).

3. The apparatus of claim 1, wherein, when the short-term training mode is selected, the display unit displays at least one image indicating at least one event that the user has a chance of encountering and displays at least one content mapped into an image selected from among the at least one displayed image according to the user input received through the external-input reception unit.

4. The apparatus of claim 3, further comprising:
a biometric-information acquisition unit configured to acquire the biometric information of the user while the content is displayed; and
an analysis unit configured to analyze the acquired biometric information,
wherein the at least one content mapped into the selected image is intended to train the user's non-cognitive ability and variably provided according to a result of the analysis of the biometric information.

5. The apparatus of claim 1, further comprising:
a determination unit configured to determine a level and type of the user's non-cognitive ability; and
a training condition setting unit configured to set a training condition according to the determined level and type,
wherein, when the long-term training mode is selected, the display unit displays at least one test content for determining the level and type of the user's non-cognitive ability,
the determination unit determines the level and type of the user's non-cognitive ability based on a user input with respect to the test content received through the external-input reception unit,
the training condition setting unit sets the training condition according to the determined level and type,
the display unit displays at least one content for training the user's non-cognitive ability according to the set training condition, and
the training condition includes at least one of a training period, a kind of the displayed content, a display time, and a display scheme.

6. The apparatus of claim 5, further comprising:
a biometric-information acquisition unit configured to acquire the biometric information of the user while the content is displayed; and
an analysis unit configured to analyze the acquired biometric information,
wherein the training condition is reset by the training condition setting unit according to a result of the analysis of the biometric information, and
the at least one content for training the user's non-cognitive ability is displayed through the display unit based on the reset training condition.

7. A method of providing content for training a user's non-cognitive ability by using a user device, the method comprising:
acquiring user information of the user;
performing user authentication based on the acquired user information;
when the authentication succeeds, selecting one of a short-term training mode and a long-term training mode based on a user input and displaying at least one content according to the selected mode; and
when the authentication fails, acquiring again updated user information,
wherein the at least one content is variably displayed according to the biometric information of the user.

8. The method of claim 7, wherein the biometric information of the user comprises at least one of a heart rate, a heart rate variability (HRV), a blood pressure, a respiration rate, an electrocardiogram (ECG), a galvanic skin response (GSR), and an electroencephalogram (EEG).

9. The method of claim 7, wherein, when the short-term training mode is selected, the displaying of the content comprises:
displaying at least one image indicating at least one event that the user has a chance of encountering; and
displaying at least one content mapped into an image selected from among the at least one displayed image according to the user input.

10. The method of claim 9, further comprising:
acquiring the biometric information of the user while the content is displayed; and
analyzing the acquired biometric information,
wherein the at least one content mapped into the selected image is intended to train the user's non-cognitive ability and variably provided according to a result of the analysis of the biometric information.

11. The method of claim 7, wherein, when the long-term training mode is selected, the displaying of the content comprises:
displaying at least one test content for determining a level and type of the user's non-cognitive ability;
determining the level and type of the user's non-cognitive ability based on a user input with respect to the test content;
setting a training condition according to the determined level and type; and
displaying at least one content for training the user's non-cognitive ability according to the set training condition,
wherein the training condition includes at least one of a training period, a kind of the displayed content, a display time, and a display scheme.

12. The method of claim 11, further comprising:
acquiring the biometric information of the user while the content is displayed;
analyzing the acquired biometric information; and
resetting the training condition according to a result of the analysis of the biometric information,
wherein the at least one content for training the user's non-cognitive ability is displayed based on the reset training condition.

13. A non-transitory computer-readable recording medium storing a computer program for executing the method of claim 7.
